(19) United States
(12) Patent Application Publication
(10) Pub. No.: US 2010/0112037 A1
(43) Pub. Date: May 6, 2010

(54) S1P RECEPTOR AGONISTS FOR THE TREATMENT OF CEREBRAL MALARIA

(76) Inventors: Max Bachrach, The Woodlands, TX (US); Constance Ann Marjory Finney, Toronto (CA); Kevin Charles Kain, Toronto (CA); Tamas Oravecz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 12/608,332

(22) Filed: Oct. 29, 2009

Related U.S. Application Data
(60) Provisional application No. 61/109,991, filed on Oct. 31, 2008.
(60) Provisional application No. 61/229,970, filed on Jul. 30, 2009.

(51) Int. Cl. A61L 15/16; A61K 31/135

(52) U.S. Cl. 424/449; 514/646

(57) ABSTRACT

Methods and compositions for treating, managing, and/or preventing cerebral malaria are disclosed.

This application claims priority to U.S. provisional application No. 61/109,991, filed Oct. 31, 2008, and U.S. provisional application 61/229,970, filed Jul. 30, 2009, the entireties of which are incorporated herein by reference.

### 1. FIELD OF THE INVENTION

This application is directed to methods of treating, managing, and/or preventing cerebral malaria, and compositions useful therein.

### 2. BACKGROUND

### 2.1. Cerebral Malaria

More than two million people, most of whom are African children, die each year of malaria. Golenser, J., et al., Int. J. Parasitology 36:583-593, 583 (2006). Eradication of the disease “has been hampered by the development of Plasmodium (especially Plasmodium falciparum, the most abundant and dangerous causative species) resistant to currently available anti-malarial drugs.” Id.

One of the most severe complications of P. falciparum infection is cerebral malaria (CM), which is expressed in about 7 percent of P. falciparum malaria cases. CM manifests as coma (Blantyre coma scale ≦2 or Glasgow coma scale ≦8), P. falciparum on blood smear, and no other known cause for coma. John, C. C., et al., Pediatrics 122:e92-e99 (2008). CM affects an estimated 785,000 children in sub-Saharan Africa every year, with an average mortality rate of 18.6 percent. Golenser at 586; John at e93. A recent study found that one in four children who survive CM suffer long-term cognitive impairment. John, id.

Although the pathogenesis of CM is unclear, a simplified explanation is that the adherence “to endothelial cells and the sequestration of parasitized erythrocytes and immune cells in brain capillaries cause an inflammatory process and the release of other neurotoxic molecules.” Golenser at 584. It is possible to treat some CM cases with anti-malaria drugs. Id. at 586. But there is an “irreversible stage after which the patient dies, despite massive anti-parasitic treatment.” Id. Thus, a number of adjunctive treatments have been suggested, some of which have shown promise, but many of which have not. See, id. at 586-591.

### 2.2. S1P Pathway

Sphingosine-1-phosphate (S1P) is a bioactive molecule with potent effects on multiple organ systems. Saba, J. D. and Hla, T. Circ. Res. 94:724-734 (2004). The compound binds with low affinity to five related G-protein coupled receptors, S1P1-5, formerly termed endothelial differentiation gene (EDG) receptor-1, -5, -3, -6, and -8, respectively. Brinkmann, V., Pharmacol. & Therapeutics 115:84-105, 85 (2007). The receptor subtypes S1P1, S1P2, and S1P3 are widely expressed in the cardiovascular system. Id. at 85-86. S1P1 is the dominant receptor on lymphocytes, and regulates their egress from secondary lymphatic organs. Id.

Numerous agonists of the S1P receptors have been reported and proposed as potential therapies in diseases that include host-versus-graft disease, rheumatoid arthritis and multiple sclerosis (MS). The S1P1 agonist FTY720 (fingolimod) in particular has been extensively studied, and is currently in clinical trials for the treatment of MS. Id. at 95-100.

It appears possible to treat some diseases by affecting other parts of the S1P pathway, as well. For example, an inhibitor of the enzyme S1P lyase, which catalyzes the cleavage of S1P into ethanolamine phosphate and a long-chain aldehyde, is effective in rheumatoid arthritis models, and is currently in clinical trials. Oravecz, T. et al., “Sphingosine-1-Phosphate Lyase is a Potential Therapeutic Target in Autoimmune Diseases Including Rheumatoid Arthritis,” Presentation 1833, American College of Rheumatology Scientific Meeting (San Francisco, Oct. 28, 2008); Pappas, C., et al., “LX2931: A Potential Small Molecule Treatment for Autoimmune Disorders,” Presentation 351, American College of Rheumatology Scientific Meeting (San Francisco, Oct. 26, 2008). See also U.S. patent application publication no. 2007/0208063; U.S. patent application Ser. No. 12/038,872.

### 3. SUMMARY OF THE INVENTION

This invention encompasses methods treating, managing, and/or preventing cerebral malaria, which comprise administering to a patient in need thereof a therapeutically or prophylactically effective amount of an S1P receptor antagonist. In some methods, the S1P receptor antagonist is administered adjunctively with one or more additional active agents.

This invention also encompasses pharmaceutical compositions useful in the treatment, management, and/or prevention of CM.

### 4. BRIEF DESCRIPTION OF THE FIGURES

Certain aspects of this invention can be understood with reference to the attached figures:

FIG. 1 shows the effect of FTY720 on the survival of mice as compared to vehicle control in the cerebral malaria model described below in the Examples.

FIG. 2 shows the effect of topical and transdermal administration of FTY720 on the white blood cell, neutrophil and lymphocyte counts of mice, measured six hours after administration. P values (Student's t test) relative to the vehicle control are shown above each histogram.

### 5. DETAILED DESCRIPTION

This invention is directed to the use of S1P receptor agonists for the treatment, management and/or prevention of cerebral malaria (CM). The invention is based, in part, on Applicants' discovery that CM may be treated by modulating the S1P pathway. For example, Applicants have discovered that both agonizing the S1P receptor and inhibiting S1P lyase can provide protection against CM in the well-established murine model of the disease. See, e.g., U.S. provisional application No. 61/109,991, filed Oct. 31, 2008, U.S. provisional application 61/229,970, filed Jul. 30, 2009, and U.S. provisional application No. 61/109,982, filed Oct. 31, 2009.

### 5.1. DEFINITIONS

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, the terms used in a description of a chemical genus taken from another cited patent or patent application are to be construed the same way as they are in that other patent or patent application.

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 5.2. S1P RECEPTOR AGONISTS

This invention encompasses compositions comprising, and methods of using, S1P receptor agonists. S1P receptor agonists are compounds that agonize one or more sphingosine-1 phosphate receptors. Preferred compounds are agonists of the S1P1 receptor.

Particular S1P receptor agonists include compounds disclosed in U.S. Pat. No. 5,604,229 to Fujita et al. These agonists include compounds of the formula:

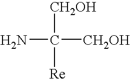

wherein Re is a phenylalkyl wherein the alkyl moiety is a straight- or branched chain having 6 to 20 carbon atoms; a phenylalkyl which may be substituted by a straight- or branched chain C6-C20 alkyl optionally substituted by halogen, a straight- or branched chain C6-C20 alkoxy optionally substituted by halogen, a straight- or branched chain C6-C20 alkenyloxy, phenylalkoxy, halophenylalkoxy, phenylalkoxyalkyl, phenoxyalkoxy or phenoxyalkyl; a cycloalkylalkyl wherein the alkyl moiety is a straight- or branched chain having 6 to 20 carbon atoms; a cycloalkylalkyl substituted by a straight- or branched chain alkyl having 6 to 20 carbon atoms; a heteroarylalkyl wherein the alkyl moiety is a straight- or branched chain having 6 to 20 carbon atoms; a heteroarylalkyl substituted by a straight- or branched chain alkyl having 6 to 20 carbon atoms; a heterocyclic alkyl wherein the alkyl moiety is a straight- or branched chain having 6 to 20 carbon atoms, or a heterocyclic alkyl substituted by a straight- or branched chain alkyl having 6 to 20 carbon atoms; wherein the alkyl moiety may have, in the carbon chain, a bond or a hereto atom selected from the group consisting of a double bond, a triple bond, oxygen, sulfur, sulfinyl, sulfonyl, —N(R⁶)— where R⁶ is hydrogen, alkyl, aralkyl, acyl or alkoxycarbonyl, and carbonyl, and may have, as a substituent, alkoxy, alkenyloxy, alkynyloxy, aralkyloxy, acyl, alkylamino, alkylthio, acylamino, alkoxycarbonyl, alkoxycarbonylamino, acyloxy, alkylcarbamoyl, nitro, halogen, amino, hydroxy or carboxy; and pharmaceutically acceptable salts thereof. See U.S. Pat. No. 5,604,229, col. 279, line 44-col. 280, line 13.

They also include compounds of the formula:

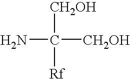

wherein Rf is a phenylalkyl wherein the alkyl moiety is a straight- or branched chain having 6 to 20 carbon atoms which may have, in the carbon chain, one or two oxygen atoms; a phenylalkyl which may be substituted by a straight- or branched chain C6-C20 alkyl optionally substituted by halogen, a straight- or branched chain C6-C20 alkoxy optionally substituted by halogen, a straight- or branched chain C6-C20 alkenyloxy, phenylalkoxy, halophenylalkoxy, phenylalkoxyalkyl, phenoxyalkoxy or phenoxyalkyl; a cycloalkylalkyl wherein the alkyl moiety is a straight- or branched chain having 6 to 20 carbon atoms which may have, in the carbon chain, one or two oxygen atoms; a cycloalkylalkyl substituted by a straight- or branched chain alkyl having 6 to 20 carbon atoms; a heteroarylalkyl wherein the alkyl moiety is a straight- or branched chain having 6 to 20 carbon atoms which may have, in the carbon chain, one or two oxygen atoms; a heteroarylalkyl substituted by a straight- or branched chain alkyl having 6 to 20 carbon atoms; a heterocyclic alkyl wherein the alkyl moiety is a straight- or branched chain having 6 to 20 carbon atoms which may have, in the carbon chain, one or two oxygen atoms, or a heterocyclic alkyl substituted by a straight- or branched chain alkyl having 6 to 20 carbon atoms; wherein the alkyl moiety may have, in the carbon chain, a substituent selected from the group consisting of alkoxy, alkenyloxy, alkynyloxy, aralkyloxy, acyl, alkylamino, alkylthio, acylamino, alkoxycarbonyl, alkoxycarbonylamino, acyloxy, alkylcarbamoyl, nitro, halogen, amino, hydroxy and carboxy; and pharmaceutically acceptable salts thereof. See U.S. Pat. No. 5,604,229, col. 280, lines 13-52.

They also include compounds of the formula:

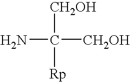

wherein Rp is a phenyl substituted by C6-C18 alkyl, a cycloalkyl, heteroaryl or a heterocycle, and pharmaceutically acceptable salts thereof. See U.S. Pat. No. 5,604,229, col. 285, lines 5-15.

They also include compounds of the formula:

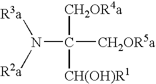

wherein R¹ is an optionally substituted straight- or branched carbon chain which may have, in the chain, a bond, a hetero atom or a group selected from the group consisting of a double bond, a triple bond, oxygen, sulfur, sulfinyl, sulfonyl, —N(R⁶)— where R⁶ is hydrogen, alkyl, aralkyl, acyl or alkoxycarbonyl, and carbonyl, optionally substituted arylene, optionally substituted cycloalkylene, optionally substituted heteroarylene and an alicycle thereof, and which may be substituted, at the chain end (w-position) thereof, by a double bond, a triple bond, optionally substituted aryl, optionally substituted cycloalkyl, optionally substituted heteroaryl or an alicycle thereof, an optionally substituted aryl, an optionally substituted cycloalkyl, an optionally substituted heteroaryl or an alicycle thereof, and R²a, R³a, R⁴a and R⁵a are the same or different and each is a hydrogen, an alkyl, an acyl or an alkoxycarbonyl; wherein the optionally substituted straight- or branched carbon chain may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxyimino, hydroxy, carboxy, optionally substituted aryl, optionally substituted aryloxy, optionally substituted cycloalkyl, optionally substituted heteroaryl and an alicycle thereof; and the aforementioned optionally substituted arylene, optionally substituted cycloalkylene, optionally substituted heteroarylene, an alicycle thereof, optionally substituted aryl, optionally substituted aryloxy, optionally substituted cycloalkyl, optionally substituted heteroaryl and an alicycle thereof may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkyl carbamoyl, haloalkyl, haloalkoxy, nitro, halogen, amino, hydroxy and carboxy; and pharmaceutically acceptable salts thereof. See U.S. Pat. No. 5,604,229, col. 285, line 33-col. 286, line 11.

They also include compounds of the formula:

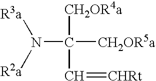

wherein Rt is an optionally substituted straight- or branched carbon chain which may have, in the chain, a bond, a hetero atom or a group selected from the group consisting of a double bond, a triple bond, oxygen, sulfur, sulfinyl, sulfonyl, —N(R⁶)— where R⁶ is hydrogen, alkyl, aralkyl, acyl or alkoxycarbonyl, carbonyl, optionally substituted arylene, optionally substituted cycloalkylene, optionally substituted heteroarylene and an alicycle thereof, an optionally substituted aryl, an optionally substituted cycloalkyl, an optionally substituted heteroaryl or an alicycle thereof, and R²a, R³a, R⁴a and R⁵a are the same or different and each is a hydrogen, an alkyl, an acyl or an alkoxycarbonyl; wherein the optionally substituted straight- or branched carbon chain may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy, carboxy, optionally substituted aryl, optionally substituted aryloxy, optionally substituted cycloalkyl, optionally substituted heteroaryl and an alicycle thereof; and the aforementioned optionally substituted arylene, optionally substituted cycloalkylene, optionally substituted heteroarylene, an alicycle thereof, optionally substituted aryl, optionally substituted aryloxy, optionally substituted cycloalkyl, optionally substituted heteroaryl and an alicycle thereof may have a substituent selected from the group consisting of alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy; and pharmaceutically acceptable salts thereof. See U.S. Pat. No. 5,604,229, col. 287, lines 1-47.

They also include compounds of the formula:

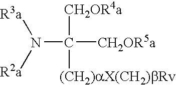

wherein Rv is an optionally substituted aryl, an optionally substituted cycloalkyl, an optionally substituted heteroaryl or an alicycle thereof; R²a, R³a, R⁴a and R⁵a are the same or different and each is a hydrogen, an alkyl, an acyl or an alkoxycarbonyl; X is an oxygen, a sulfur, a sulfinyl, a sulfonyl, —N(R⁶)— where R⁶ is hydrogen, alkyl, aralkyl, acyl or alkoxycarbonyl; and α and β are 0 or an integer of 1-20 provided that α+β=5-20, wherein the optionally substituted aryl, optionally substituted cycloalkyl, optionally substituted heteroaryl and an alicycle thereof may have a substituent selected from the group consisting of alkyl, alkoxy, alkenyloxy, alkynyloxy, aralkyloxy, alkylenedioxy, acyl, alkylamino, alkylthio, acylamino, alkoxycarbonyl, alkoxycarbonylamino, acyloxy, alkylcarbamoyl, haloalkyl, haloalkoxy, nitro, halogen, amino, hydroxy and carboxy; and pharmaceutically acceptable salts thereof. See U.S. Pat. No. 5,604,229, col. 288, lines 1-28.

S1P receptor agonists include compounds disclosed in U.S. Pat. No. 5,719,176 to Fujita et al. These agonists include compounds of the formula:

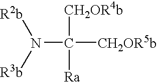

wherein Ra is a straight- or branched chain alkyl having 12 to 22 carbon atoms, which may have, in the chain, a bond or a hetero atom selected from the group consisting of a double bond, a triple bond, oxygen, sulfur, sulfinyl, sulfonyl, —N(R⁶)— where R⁶ is hydrogen, alkyl, aralkyl, acyl or alkoxycarbonyl, and carbonyl, and which may have, as a substituent, alkoxy, alkenyloxy, alkynyloxy, aralkyloxy, acyl, alkylamino, alkylthio, acylamino, alkoxycarbonyl, alkoxycarbonylamino, acyloxy, alkylcarbamoyl, nitro, halogen, amino, hydroxyimino, hydroxy or carboxy, and R²b, R³b, R⁴b and R⁵b are the same or different and each is a hydrogen, an alkyl or an acyl; and pharmaceutically acceptable salts thereof. See U.S. Pat. No. 5,719,176, col. 274, line 48-col. 275, line 3.

S1P receptor agonists include compounds disclosed in U.S. Pat. No. 5,948,820 to Fujita et al. These agonists include compounds of the formula:

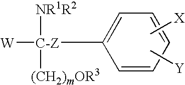

wherein W is hydrogen; a straight- or branched chain alkyl having 1 to 6 carbon atoms; a straight- or branched chain alkenyl having 2 to 6 carbon atoms; a straight- or branched chain alkynyl having 2 to 6 carbon atoms; or a straight- or branched chain C1-C6 alkyl substituted by 1 to 3 substituents selected from the group consisting of a halogen, a cycloalkyl and a phenyl which may be substituted by hydroxy; X is a straight-chain alkyl having carbon atoms in the number of p or a straight-chain alkoxy having carbon atoms in the number of (p-1), wherein the straight-chain alkyl having carbon atoms in the number of p and the straight-chain alkoxy having carbon atoms in the number of (p-1) may have 1 to 3 substituents selected from the group consisting of an alkyl, hydroxy, an alkoxy, an acyloxy, amino, an alkylamino, an acylamino, oxo, a haloalkyl, a halogen and a phenyl which may have 1 to 3 substituents selected from the group consisting of an alkyl, hydroxy, an alkoxy, an acyl, an acyloxy, amino, an alkylamino, an acylamino, a haloalkyl and a halogen; Y is hydrogen, an alkyl, hydroxy, an alkoxy, an acyl, an acyloxy, amino, an alkylamino, an acylamino, a haloalkyl or a halogen; Z is a straight-chain alkylene having carbon atoms in the number of q; p and q are the same or different and each is an integer of 1 to 20, with the proviso of 6≦p+q≦23; m is 1, 2 or 3; n is 2 or 3; R¹ and R² are the same or different and each is hydrogen, an alkyl or an acyl; R³ is hydrogen or an acyl; and pharmaceutically acceptable salts thereof. See U.S. Pat. No. 5,948,820, col. 164, lines 14-56.

S1P receptor agonists include compounds disclosed in U.S. Pat. No. 6,214,873 to Kunitomo et al. These agonists include compounds of the formula:

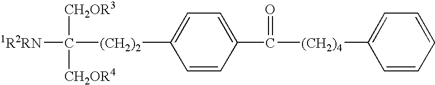

wherein R¹, R², R³ and R⁴ are the same or different and each is a hydrogen or an acyl, and pharmaceutically acceptable salts thereof. See U.S. Pat. No. 6,214,873, col. 54, lines 50-63.

S1P receptor agonists include compounds disclosed in U.S. Pat. No. 6,437,165 to Mandala et al. These agonists include compounds of the formula:

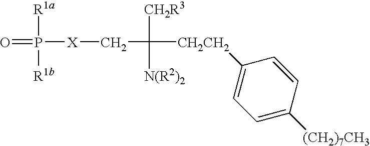

wherein X is O, S, NR¹ or (CH₂)₁₋₂, optionally substituted with 1-4 halo groups; R¹ is H, C₁₋₄alkyl or haloC₁₋₄alkyl; R^{1a} is H, OH, C₁₋₄alkyl, or OC₁₋₄alkyl, the alkyl and alkyl portions being optionally substituted with 1-3 halo groups; R^{1b} represents H, OH, C₁₋₄alkyl or haloC₁₋₄alkyl; R² is H, C₁₋₄alkyl or haloC₁₋₄alkyl, and R³ is H, OH, halo, OC₁₋₄alkyl or O-haloC₁₋₄alkyl, and pharmaceutically acceptable salts thereof. See U.S. Pat. No. 6,437,165, col. 25, lines 42-63.

S1P receptor agonists include compounds disclosed in U.S. Pat. No. 6,723,745 to Nishi et al. These agonists include compounds of the formula:

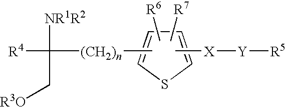

wherein R¹ and R² are the same or different and each represents a hydrogen atom or an amino protecting group; R³ represents a hydrogen atom or a hydroxy protecting group; R⁴ represents a lower alkyl group; n represents an integer from 1 to 6; X represents an ethylene group, a vinylene group, an ethynylene group, a group of formula -D-CH₂— (wherein D represents a carbonyl group, a group of formula —CH(OH)—, an oxygen atom, a sulfur atom, or a nitrogen atom), an aryl group, or an aryl group substituted with 1 to 3 substituents selected from substituent group a; Y represent a single bond, a C₁-C₁₀ alkylene group, a C₁-C₁₀ alkylene group substituted with 1 to 3 substituents selected from substituent groups a and b, a C₁-C₁₀ alkylene group which has an oxygen atom or a sulfur atom in said carbon chain or at the end of said carbon chain, or a C₁-C₁₀ alkylene group which is substituted with 1 to 3 substituents selected from substituent groups a and b and has an oxygen atom or a sulfur atom in said carbon chain or at the end of said carbon chain; R⁵ represents a hydrogen atom, a cycloalkyl group, an aryl group, a heterocyclic group, a cycloalkyl group substituted with 1 to 3 substituents selected from substituent groups a and b, an aryl group substituted with 1 to 3 substituents selected from substituent groups a and b, or a heterocyclic group substituted with 1 to 3 substituents selected from substituent groups a and b; R⁶ and R⁷ are the same or different and each represent a hydrogen atom or a group selected from substituent group a; with the proviso that when R⁵ is a hydrogen atom, Y is not a single bond or a straight chain C₁-C₁₀ alkylene group; substituent group a consists of a halogen atom, a lower alkyl group, a halogenated lower alkyl group, a lower alkoxy group, a lower alkylthio group, a carboxyl group, a lower alkoxycarbonyl group, a hydroxyl group, a lower aliphatic acyl group, an amino group, a mono lower alkylamino group, a di lower alkylamino group, a lower aliphatic acylamino group, a cyano group, and a nitro group; substituent group b consists of a cycloalkyl group, an aryl group, a heterocyclic group, a cycloalkyl group substituted with 1 to 3 substituents selected from substituent group a, an aryl group substituted with 1 to 3 substituents selected from substituent group a, and a heterocyclic group substituted with 1 to 3 substituents selected from substituent group a. See U.S. Pat. No. 6,723,745, col. 222, line 41-col. 223, line 34.

S1P receptor agonists include compounds disclosed in U.S. Pat. No. 6,963,012 to Kohno et al. These agonists include compounds of the formula:

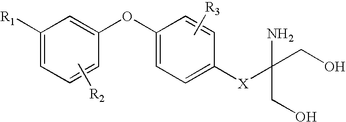

wherein R₁ is halogen, trihalomethyl, hydroxy, lower alkyl having 1 to 7 carbon atoms, phenyl, aralkyl, lower alkoxy having 1 to 4 carbon atoms, trifluoromethyloxy, substituted or unsubstituted phenoxy, cyclohexylmethyloxy, substituted or unsubstituted aralkyloxy, pyridylmethyloxy, cinnamyloxy, naphthylmethyloxy, phenoxymethyl, hydroxymethyl, hydroxyethyl, lower alkylthio having 1 to 4 carbon atoms, lower alkylsulfinyl having 1 to 4 carbon atoms, lower alkylsulfonyl having 1 to 4 carbon atoms, benzylthio, acetyl, nitro, or cyano; R₂ is hydrogen, halogen, trihalomethyl, lower alkoxy having 1 to 4 carbon atoms, lower alkyl having 1 to 7 carbon atoms, phenethyl, or benzyloxy; R₃ is hydrogen, halogen, trifluoromethyl, lower alkoxy having 1 to 4 carbon atoms, hydroxy, benzyloxy, lower alkyl having 1 to 7 carbon atoms, phenyl, lower alkoxymethyl having 1 to 4 carbon atoms, or lower alkylthio having 1 to 4 carbon atoms; and X is —(CH₂)ₙ— (n is an integer from 1 to 4), —OCH₂CH₂—, or CH═CHCH₂—. See U.S. Pat. No. 6,963,012, col. 60, lines 36-65.

S1P receptor agonists include compounds disclosed in U.S. Pat. No. 7,241,812 to Saha et al. These agonists include compounds of the formula:

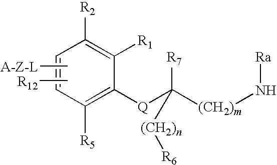

wherein L is alkoxy, a covalent bond, substituted or unsubstituted alkyl, alkylcarbonyl, thioether, alkylsulfonyl, alkylcarbonylamino, alkylaminocarbonyl, alkyloxycarbonyl, alkylcarbonyloxy, or substituted or unsubstituted heteroaryl; Z and A are each independently substituted or unsubstituted aryl, wherein Z and A may be linked by a covalent bond, substituted or unsubstituted alkyl, NH, alkyloxy, O, thioether, S, aminocarbonyl, carbonylamino, carbonyloxy, or oxycarbonyl; R₁, R₂, R₅ and R₁₂ are each independently selected from the group consisting of hydrogen, halogen, cyano, substituted or unsubstituted aryl, straight chain or branched substituted or unsubstituted C₁-C₆-alkyl, straight chain or branched substituted or unsubstituted C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched substituted or unsubstituted C₁-C₆-alkyl, straight chain or branched substituted or unsubstituted C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; Q is —NH(CO)—; R₆ is —OPO₃R₁₀R₁₁, where R₁₀ and R₁₁ are each independently H, straight chain or branched substituted or unsubstituted C₁-C₆-alkyl, a substituted or unsubstituted aryl group or selected from the prodrugs listed below:

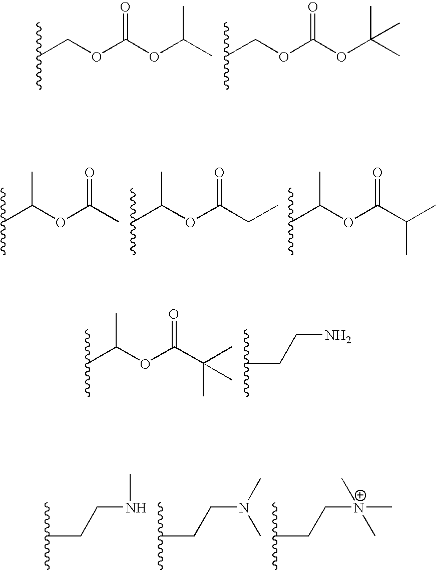

R₇ is H, substituted or unsubstituted C₁-C₆-alkyl, hydroxy-C₁-C₆-alkyl, aryl, or together with R₈ form a C₂-C₅-alkylene or a C₂-C₅-alkenylene group; R₈ is H or substituted or unsubstituted C₁-C₆-alkyl; and m and n are each, independently, an integer from 0 to 3; and pharmaceutically acceptable salts thereof. See U.S. Pat. No. 7,241,812, col. 169, line 2-col. 170, line 37.

S1P receptor agonists include compounds disclosed in U.S. Pat. No. 7,326,801 to Albert et al. These agonists include compounds of the formula:

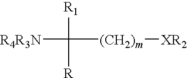

wherein m is 1, 2 or 3; X is OR₁ is H; C₁₋₆ alkyl optionally substituted by OH, acyl, halogen, cycloalkyl, phenyl or hydroxy-phenylene; C₂₋₆alkenyl; C₂₋₆alkynyl; or phenyl optionally substituted by OH; R₂ is

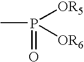

wherein R₅ is H or C₁₋₄alkyl optionally substituted by 1, 2 or 3 halogen atoms, and R₆ is H or C₁₋₄alkyl optionally substituted by halogen; each of R₃ and R₄, independently, is H, C₁₋₄alkyl optionally substituted by halogen, or acyl, and R is a residue of the formula

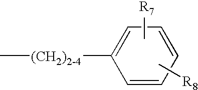

wherein R₇ is H, C₁₋₄alkyl or C₁₋₄alkoxy, and R₈ is (a) C₁₋₂₀alkanoyl or C₁₋₁₄alkoxy substituted with cycloalkyl or phenyl wherein the cycloalkyl or phenyl ring is optionally substituted by halogen, C₁₋₄alkyl and/or C₁₋₄alkoxy, (b) phenylC₁₋₁₄-alkyl wherein the C₁₋₁₄alkyl is optionally substituted by halogen or OH, (c) cycloalkylC₁₋₁₄alkoxy or phenylC₁₋₁₄alkoxy wherein the cycloalkyl or phenyl ring is optionally substituted by halogen, C₁₋₄alkyl and/or C₁₋₄alkoxy, or (d) phenylC₁₋₄alkoxyC₁₋₄alkyl, phenoxyC₁₋₁₄alkoxy or phenoxyC₁₋₄alkyl, and pharmaceutically acceptable salts thereof. See U.S. Pat. No. 7,326,801, col. 25, line 12-col. 26, line 22.

S1P receptor agonists include compounds disclosed in U.S. patent application publication no. 2005/0033055 to Bugianesi et al. These agonists include compounds of the formula:

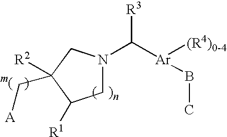

wherein Ar is phenyl or naphthyl; m=0 or 1; n=0 or 1; A is selected from the group consisting of: —CO₂H, —PO₃H₂, —PO₂H, —SO₃H, —PO(C₁₋₃alkyl)OH and 1H-tetrazol-5-yl; R¹ and R² are each independently selected from the group consisting of: hydrogen, halo, hydroxy, —CO₂H and C₁₋₄alkyl, optionally substituted from one up to the maximum number of substitutable positions with halo; R₃ is selected from the group consisting of: hydrogen and C₁₋₄alkyl, optionally substituted with from one up to the maximum number of substitutable positions with a substituent independently selected from the group consisting of: halo and hydroxy; each R⁴ is independently selected from the group consisting of: halo, C₁₋₄alkyl and C₁₋₃alkoxy, said C₁₋₄alkyl and C₁₋₃alkoxy optionally substituted from one up to the maximum number of substitutable positions with halo, C is selected from the group consisting of: (1) C₁₋₈alkyl, C₁₋₈alkoxy, —(C═O)—CC₁₋₆alkyl or —CHOH—C₁₋₆alkyl, said C₁₋₈alkyl, C₁₋₈alkoxy, —(C═O)—C₁₋₆alkyl and —CHOH—C₁₋₆alkyl optionally substituted with phenyl, and (2) phenyl or HET, each optionally substituted with 1-3 substituents independently selected from the group consisting of: halo, phenyl, C₁₋₄alkyl and C₁₋₄alkoxy, said C₁₋₄alkyl and C₁₋₄alkoxy groups optionally substituted from one up to the maximum number of substitutable positions with a substituent independently selected from halo and hydroxy, and said phenyl optionally substituted with 1 to 5 groups independently selected from the group consisting of: halo and C₁₋₄alkyl, optionally substituted with 1-3 halo groups, or C is not present; when C is not present then B is selected from the group consisting of: phenyl, C₅₋₁₆alkyl, C₅₋₁₆alkenyl, C₅₋₁₆alkynyl, —CHOH—C₄₋₁₅alkyl, —CHOH—C₄₋₁₅alkenyl, —CHOH—C₄₋₁₅alkynyl, C₄₋₁₅alkoxy, —OC₄₋₁₅alkenyl, —OC₄₋₁₅alkynyl, C₄₋₁₅alkylthio, —SC₄₋₁₅alkenyl, —SC₄₋₁₅alkynyl, —CH₂C₃₋₁₄alkoxy, —CH₂OC₃₋₁₄alkenyl, —CH₂OC₃₋₁₄alkynyl, —(C═O)C₄₋₁₅alkyl, —(C═O)C₄₋₁₅alkenyl, —(C═O)—C₄₋₁₅alkynyl, —(C═O)OC₃₋₁₄alkyl, —(C═O)OC₃₋₁₄alkenyl, —(C═O)N(R⁶)(R⁷)C₃₋₁₄alkyl, —(C═O)N(R⁶)(R⁷)C₃₋₁₄alkenyl, —(C═O)N(R⁶)(R⁷)C₃₋₁₄alkynyl, —N(R⁶)(R⁷)(C═O)C₃₋₁₄alkyl, —N(R⁶)(R⁷)(C═O)C₃₋₁₄alkenyl and —N(R⁶)(R⁷)(C═O)C₃₋₁₄alkynyl, when C is phenyl or HET then B is selected from the group consisting of: C₁₋₆alkyl, C₁₋₅alkoxy, —(C═O)C₁₋₅alkyl, —(C═O)OC₁₋₄alkyl, —(C═O)N(R⁶)(R⁷)C₁₋₄alkyl, phenyl and HET, and when C is C₁₋₈alkyl, C₁₋₈alkoxy, —(C═O)C₁₋₆alkyl or —CHOHC₁₋₆alkyl then B is phenyl; and R⁶ and R⁷ are independently selected from the group consisting of: hydrogen, C₁₋₉alkyl and —(CH₂)ₚ-phenyl, wherein p is 1 to 5 and phenyl is optionally substituted with 1-3 substituents independently selected from the group consisting of: C₁₋₃alkyl and C₁₋₃alkoxy, each optionally substituted with 1-3 halo groups; and pharmaceutically acceptable salts thereof. See U.S. patent application publication no. 2005/0033055, pages 47-48. In this context, the term HET refers to moieties selected from the group consisting of:

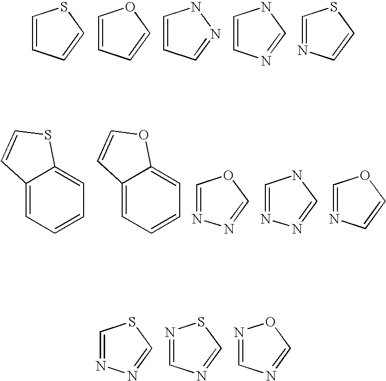

Id. at paragraph 0041.

S1P receptor agonists include compounds disclosed in international patent application no. WO 2006/088944 to Lynch et al. These agonists include compounds of the formulae:

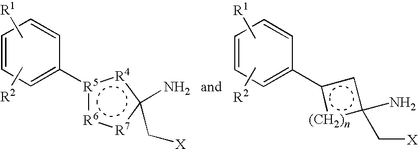

wherein R⁴ and R⁷ are independently CH or CH₂; R⁵ is C, CH or N; R⁶ is CH, CH₂, O, S or NR³, wherein R³ is hydrogen or a (C₁-C₁₀) alkyl group; X is selected from hydroxyl, phosphate, phosphonate, alpha-substituted phosphonate; R¹ is selected from the group consisting of hydrogen, halo, trifluoromethyl, (C₁-C₁₀) alkyl, (C₁-C₁₀) alkyl substituted with halo, hydroxyl, (C₁-C₁₀) alkoxy, or cyano; and R² is selected from the group consisting of (C₁-C₂₀) alkyl, cycloalkyl substituted alkyl, (C₁-C₂₀)alkenyl, (C₁-C₂₀)alkynyl, aryl, alkyl substituted aryl, arylalkyl and aryl substituted arylalkyl; wherein one or more of the carbon atoms in the R² groups can be independently replaced with non-peroxide oxygen, sulfur or NR⁸; wherein R⁸ is hydrogen or a (C₁-C₁₀) alkyl group; wherein the alkyl, alkenyl and alkynyl groups in R² are optionally substituted with oxo, n is 0, 1, 2 or 3; and the dashed circle represents 1, 2 or 3 optional double bonds, and pharmaceutically acceptable salts thereof. See WO 2006/088944 at page 45.

S1P receptor agonists include compounds disclosed in international patent application no. WO 2008/029371 to Bolli et al. These agonists include compounds of the formula:

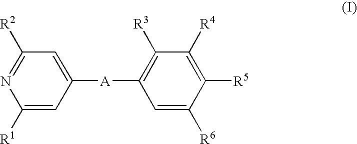

wherein A represents *—CONHCH₂—, *—CO—CH═CH—, *—COCH₂CH₂—,

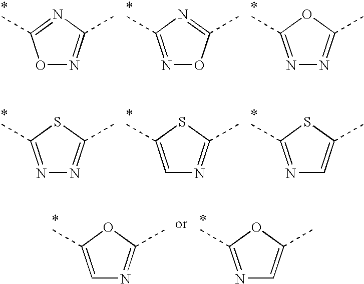

wherein the asterisks indicate the bond that is linked to the pyridine group of Formula (I); R¹ represents C₁₋₄-alkyl or chloro; R² represents C₁₋₅-alkyl, C₁₋₄-alkoxy, or C₃₋₆-cycloalkyl; R³ represents hydrogen, C₁₋₄-alkyl, C₁₋₄-alkoxy, or halogen; R⁴ represents hydrogen, C₁₋₄-alkyl, C₁₋₄-alkoxy, halogen, trifluoromethyl, or trifluoromethoxy; R⁵ represents 2,3-dihydroxypropyl, di-(hydroxy-C₁₋₄-alkyl)-C₁₋₄-alkyl, —CH₂—(CH₂)ₖ—NHSO₂R⁵³, —(CH₂)ₙ—CH(OH)CH₂NHSO₂R⁵³, —CH₂(CH₂)ₖ—NHCOR⁵⁴, —(CH₂)CH(OH)CH₂—NHCOR⁵⁴, —CH²—(CH₂)ₙ—CONR⁵¹R⁵², —CO—NHR⁵¹, 1-(3-carboxy-azetidinyl)-2-acetyl, 1-(2-carboxy-pyrrolidinyl)-2-acetyl, 1-(3-carboxy-pyrrolidinyl)-2-acetyl, 1-(3-carboxy-azetidinyl)-3-propionyl, 1-(2-carboxy-pyrrolidinyl)-3-propionyl, 1-(3-carboxy-pyrrolidinyl)-3-propionyl, —(CH₂)—CH(OH)—CH₂—NR⁵¹R⁵², hydroxy, hydroxy-C₂₋₅-alkoxy, di-(hydroxy-C₁₋₄-alkyl)-C₁₋₄-alkoxy, 2,3-dihydroxy-propoxy, 2-hydroxy-3-methoxy-propoxy, —OCH₂—(CH₂)ₘ—NR⁵¹R⁵², 2-[((azetidine-3-carboxylic acid)-1-yl]-ethoxy, 2-[(azetidine-3-carboxylic acid C₁₋₅-alkylester)-1-yl]-ethoxy, 2-[(pyrrolidine-3-carboxylic acid)-1-yl]-ethoxy, 2-[(pyrrolidine-3-carboxylic acid C₁₋₅-alkylester)-1-yl]-ethoxy, —OCH₂—CH(OH)—CH²—NR⁵¹R⁵², 3-[(azetidine-3-carboxylic acid)-1-yl]-2-hydroxypropoxy, 3-[(azetidine-3-carboxylic acid C₁₋₅-alkylester)-1-yl]-2-hydroxypropoxy, 2-hydroxy-3-[(pyrrolidine-3-carboxylic acid)-1-yl]-propoxy, 2-hydroxy-3-[(pyrrolidine-3-carboxylic acid C₁₋₅-alkylester)-1-yl]-propoxy, 2-hydroxy-3-[(pyrrolidine-2-carboxylic acid)-1-yl]-propoxy, 2-hydroxy-3-[(pyrrolidine-2-carboxylic acid C₁₋₅-alkylester)-1-[-propoxy, —OCH₂—(CH₂)ₘ—NHSO₂R⁵³, —OCH₂—CH(OH)—CH₂—NHSO₂R⁵³, —OCH₂—(CH₂)ₘ—NHCOR⁵⁴, —OCH₂—CH(OH)—CH₂—NHCOR⁵⁴; R⁵¹ represents hydrogen, C₁₋₃-alkyl, 2-hydroxyethyl, 2-hydroxy-1-hydroxymethyl-ethyl, 2,3-dihydroxypropyl, carboxymethyl, 1-(C₁₋₅-alkylcarboxy)methyl, 2-carboxyethyl, or 2-(C₁₋₅-alkylcarboxy)ethyl; R⁵² represents hydrogen, methyl, or ethyl; R⁵³ represents C₁₋₃-alkyl, methylamino, ethylamino, or dimethylamino; R⁵⁴ represents hydroxymethyl, hydroxyethyl, aminomethyl, methylaminomethyl, dimethylaminomethyl, aminoethyl, 2-methylamino-ethyl, or 2-dimethylamino-ethyl; k represents the integer 1, 2, or 3; m represents the integer 1 or 2; n represents 0, 1, or 2; and R⁶ represents hydrogen, C₁₋₄-alkyl, or halogen; and salts thereof. See WO 2008/029371 at pages 117-118.

S1P receptor agonists also include compounds disclosed in: international patent application no. WO 2008/035239 to Bolli et al.; U.S. patent application publication no. 2008/0064662 to Saha et al., and; U.S. patent application publication no. 2008/0070866 to Deng et al.

Specific S1P receptor agonists include S1P itself, SEW2871, JTE-013, VPC23019, R-3477 (Actelion), KRP-203 (Kyorin Pharmaceutical Co.), sonepcizumab (Lpath), BAF-312 (Novartis), ONO-4641 (Ono Pharmaceutical Co.), ES-285 (PharmaMar SA), 2-amino-2-[2-(4-octylphenyl)ethyl]propane-1,3-diol (FTY720; fingolimod), phospho-FTY720, and pharmaceutically acceptable salts thereof.

### 5.3. ADDITIONAL ACTIVE AGENTS

Some embodiments of the invention employ one or more active agents in addition to an S1P receptor agonist. Examples of such additional agents include anti-malarial drugs (e.g., quinine, quinidine, and artemisinin derivatives such as artemether and artesunate), osmotic diuretics (e.g., mannitol and urea), anti-convulsants (e.g., diazepam, phenyloin, phenobarbital, and phenobarbitone), anti-pyretics (e.g., paracetamol), anti-oxidants, and anti-inflammatory drugs (e.g., NSAIDS, steroids, cyclosporin, thalidomide, revlimid, anti-TNF antibodies (e.g., infliximab, etanercept), and pentoxifylline). Others include curdlan sulfate, curcumin, and LMP-420.

### 5.4. METHODS OF USE

This invention encompasses methods of preventing, managing and treating CM, which comprise administering to a patient a therapeutically or prophylactically effective amount of an S1P receptor agonist. The amount of drug, dosing schedule, and route of administration will vary depending on the drug and the patient, and can readily be determined by those of ordinary skill in the art. Because oral administration of drugs may be difficult in some CM patients, preferred routes of administration include i.v. and i.m.

In some embodiments of the invention, the S1P receptor agonist is administered adjunctively with one or more additional active agents. Administration of the two or more drugs may be concurrent (e.g., in the same dosage form, or in separate dosage forms administered to the patient at approximately the same time), but need not be.

Methods of treating and managing CM are suitable for patients exhibiting one or more symptoms of CM, including coma (Blantyre coma scale ≦2 or Glasgow coma scale ≦8), P. falciparum on blood smear, and no other known cause for coma. Methods of preventing CM are suitable for patients at risk of CM, e.g., patients having P. falciparum on blood smear and optionally exhibiting one or more additional symptoms of malaria, including those of severe malaria (e.g., severe malarial anemia, respiratory distress, shock, spontaneous bleeding, hypoglycemia, repeated seizures, hemoglobinuria, hypoglycemia, prostration, impaired consciousness, jaundice, hyperparasitemia). Patients include adults and children (e.g., ages 5-12 years).

### 5.5. PHARMACEUTICAL FORMULATIONS

Pharmaceutical compositions include single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed. (Mack Publishing, Easton Pa.: 1990).

5.5.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See, e.g., Remington's Pharmaceutical Sciences, 18^{th} ed. (Mack Publishing, Easton Pa.: 1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. Liquid oral dosage forms are preferred for most patients suffering from CM.

5.5.2. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

5.5.3. Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 6. EXAMPLES

Aspects of this invention can be understood from the following examples, which do not limit its scope.

6.1. Measuring S1P Receptor Binding Affinity

The binding affinity of S1P receptor agonists to individual human S1P receptors may be determined using well known assays. For example, compounds can be tested using the human S1P receptors S1P₁, S1P₂, S1P₃, S1P₄ and S1P₅ by quantifying compound induced GTP[γ-³⁵S] binding to membrane protein prepared from transfected CHO or RH7777 cells stably expressing the appropriate human S1P receptor. A suitable assay technology is SPA (scintillation proximity based assay). Briefly, DMSO dissolved compounds are serially diluted and added to SPA-bead (Amersham-Pharmacia) immobilized S1P receptor expressing membrane protein (10-20 μg/well) in the presence of 50 mM Hepes, 100 mM NaCl, 10 mM MgCl₂, 10 μM GDP, 0.1% fat free BSA and 0.2 nM GTP[γ-³⁵S] (1200 Ci/mmol). After incubation in 96 well microtiterplates at RT for 120 minutes, unbound GTP[γ-³⁵S] is separated by a centrifugation step. Luminescence of SPA beads triggered by membrane bound GTP[γ-³⁵S] is quantified with a TOPcount plate reader (Packard). EC₅₀s are calculated using standard curve fitting software.

Internalization and desensitization of S1P receptors can be determined using, for example, CHO cells transfected with a myc-tagged human S1P receptor. Internationalization of the receptor as a results of stimulation by agonists is determined by FACS analysis using fluorescently labeled anti-myc antibodies.

6.2. Cerebral Malaria Model

A well characterized and widely used animal model of CM was used to test the efficacy of compounds. See, e.g., Golenser, supra, at 585. In each experiment, two groups of C56B1/6 mice were infected with 1 million parasites (P. berghei ANKA) i.p. in 500 μl of media. The first group was the control group, and the second was treated daily by gavage with FTY720 (0.3 mg/kg/day).

After at least two doses of the drug had been administered, tail vein blood was taken from the mice, and flow cytometry analysis was used to assess the levels of B and T cells, using antibodies to CD3, CD4, CD8 and CD19. The animals were monitored daily for body weight and parasitaemia, and twice daily for survival.

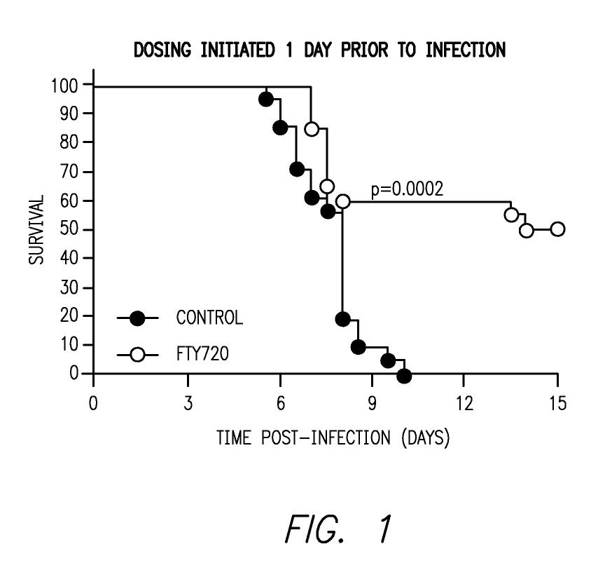

As shown in FIG. 1, FTY720 provided a significant survival advantage if administered one day prior to malaria infection (two independent experiments, n=10 per group minimum, Log-Rank Test, p=0.0002).

6.3. Transdermal Patch

Drug-in-adhesive transdermal patches containing FTY720 were made by dissolving FTY720 in adhesive (Duro-Tak 87-2196, National Starch & Chemical Co.) at a ratio of 1 part compound to 10 parts adhesive (weight:weight). Adhesive containing FTY720 was layered onto a release liner (Scotchpak 1022 PET Film, 3M Corporation) using a Bird applicator with a 50 to 200 micron gap. Organic solvents were removed from the film by baking at 100° C. for 15 minutes. The dried adhesive was then laminated onto a backing membrane (CoTran 9720 polyethylene film, 3M Corporation).

6.4. Topical and Transdermal Administration

Topical and transdermal dosage forms of FTY720 were administered to F1 hybrid mice (n=5 mice per group). The blood of the mice was collected at various time points for CBC and PK analysis.

Topical dosing of FTY720 was achieved using 200 μL of a 1 mg/mL solution composed of 70% ethanol, 29.9% water, and 0.1% DMSO.

Transdermal dosing was achieved using patches made as described above. Patches were cut from the laminate and applied to bare skin above the front shoulder of the mice. Dosage was controlled by adjusting the thickness of the compound/adhesive applied to the release liner and number or size of patches. Fur was trimmed from the site of application and then shaved to expose bare skin. To improve adhesion, the skin was further prepped by swabbing with 70% ethanol and then allowed to air dry prior to placing the patch.

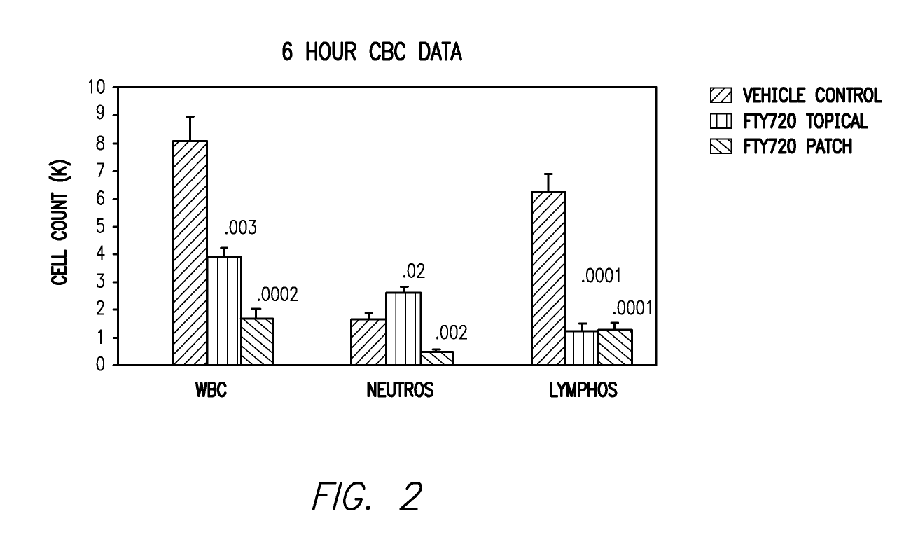

As shown in FIG. 2, FTY720 affected the white blood cell, neutrophil and lymphocyte counts of mice, measured six hours after administration.

All references (e.g., patents and patent applications) cited above are incorporated herein by reference in their entireties.

What is claimed is:

1. A method of treating, managing or preventing cerebral malaria, which comprises administering to a patient in need of such treatment, management or prevention a therapeutically or prophylactically effective amount of an S1P receptor agonist.

2. The method of claim 1, wherein the S1P receptor agonist is administered topically or transdermally.

3. The method of claim 1, wherein the S1P receptor agonist is administered intravenously.

4. The method of claim 1, wherein the S1P receptor agonist is of the formula: 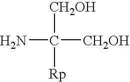
wherein Rp is a phenyl substituted by C₆-C₁₈ alkyl, a cycloalkyl, heteroaryl or a heterocycle, or a pharmaceutically acceptable salt thereof.

5. The method of claim 4, wherein the S1P receptor agonist is FTY720.

6. The method of claim 1, which further comprises administering to the patient an additional active agent.

7. The method of claim 6, wherein the additional active agent is an anti-malarial drug.

8. The method of claim 7, wherein the anti-malaria drug is quinine, quinidine, artemether or artesunate.

9. The method of claim 6, wherein the additional active agent is an osmotic diuretic.

10. The method of claim 6, wherein the additional active agent is an anti-convulsant.

11. The method of claim 6, wherein the additional active agent is an anti-pyretic.

12. The method of claim 6, wherein the additional active agent is an anti-oxidant.

13. The method of claim 6, wherein the additional active agent is an anti-inflammatory drug.

14. The method of claim 13, wherein the anti-inflammatory drug is an NSAID, steroid, cyclosporin, thalidomide, revlimid, or anti-TNF antibody.

15. The method of claim 6, wherein the additional active agent is curdlan sulfate, curcumin, or LMP-420.

16. A pharmaceutical formulation comprising an S1P receptor agonist and an additional active agent, wherein the additional active agent is an anti-malarial drug.

17. The formulation of claim 16, wherein the S1P receptor agonist is of the formula: 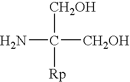
wherein Rp is a phenyl substituted by C₆-C₁₈ alkyl, a cycloalkyl, heteroaryl or a heterocycle, or a pharmaceutically acceptable salt thereof.

18. The formulation of claim 17, wherein the S1P receptor agonist is FTY720.

19. The formulation of claim 16, wherein the anti-malaria drug is quinine, quinidine, artemether or artesunate.

20. A single unit dosage form suitable for parenteral delivery, which comprises an S1P receptor agonist and an anti-malarial drug.

21. The single unit dosage form of claim 20, wherein the S1P receptor agonist is FTY720.

22. A single unit dosage form suitable for transdermal or topical delivery, which comprises an S1P receptor agonist and an anti-malarial drug.

23. The single unit dosage form of claim 22, wherein the S1P receptor agonist is FTY720.

24. The single unit dosage form of claim 22, which is a patch.
